(19) United States
(12) Patent Application Publication
(10) Pub. No.: US 2007/0020291 A1
(43) Pub. Date: Jan. 25, 2007

(54) Vaccines for protection from Bartonella infection and related methods of use

(76) Inventors: Frederick Randall Bethke, Millsboro, DE (US); Jane Kwun-Lai Kan Battles, Dagsboro, DE (US); Frank Jay Sterner, Rehoboth Beach, DE (US); Melissa Anne Lum, Rehoboth Beach, DE (US)

Correspondence Address: INTERVET INC.;PATENT DEPARTMENT, PO BOX 318, MILLSBORO, DE, 19966-0318, US

(21) Appl. No.: 11/451,931

(22) Filed: Jun. 13, 2006

Related U.S. Application Data
(60) Provisional application No. 60/690,251, filed on Jun. 14, 2005.

(51) Int. Cl. A61K 39/02

(52) U.S. Cl. 424/234.1

(57) ABSTRACT

Embodiments of the present invention generally comprise vaccines for substantially inhibiting or preventing infection from Bartonalla and related methods of vaccination.

### PRIORITY CLAIM TO RELATED PATENT APPLICATION

This patent claims priority to U.S. Provisional Patent Application No. 60/690,251 (filed Jun. 14, 2005). U.S. Provisional Patent Application No. 60/690,251 is incorporated by reference into this patent.

### FIELD OF THE INVENTION

Various embodiments of the present invention generally comprise vaccines for protection from Bartonella infection and related methods.

### BACKGROUND OF THE INVENTION

Domestic cats have been implicated as the major reservoir of B. henselae, the causative agent of cat scratch disease and a broad spectrum of acute and chronic diseases in humans.

Bartonella henselae is an agent of cat scratch disease (CSD) and has been associated with bacillary angiomatosis, bacillary peliosis, recurrent bacterimia, and endocarditis. (cat scratch disease, bacillary angiomatosis, and other infections due to Rochalimaea, New England Journal of Medicine, 1994, 330: pages 1509-1515). While cats have been shown through evidence to serve as vectors for the transmission of Bartonella henselae to people, cats may be asymptomatic to natural infection. (Bartonella (Rochalimaea) Bacterimia and Three Feline Populations, Kordick et al., abstracts of the 34 Inter Science Conference on Anti-Microbial Agents and Chemotherapy, American Society for Microbiology Washington D.C., 1994). However, some recent studies have indicated that experimentally infected cats may develop clinical signs such as fever, anorexia, lethargy, and peripheral lymphadenopathy. (Experimental and Natural Infection with Bartonella henselae in domestic cats, Comp. Immuno. Microbial. In Fact. Dis., 1997, 20:pages 41-51). These clinical signs dissipate within a short time and may not even be noticed by the cat owner. However, infections are prone to relapse.

There are conflicting reports with regard to clinical signs of experimentally infected cats. (Experimental of Natural infection with Bartonella henselae in domestic cats, Abbott et al., Comp. Immunol. Microbol. Infect. Dis., 1997, 20: pages 41-51). Various studies have reported absence of clinical signs in experimentally infected cats while others have reported mild clinical signs, including mild fever, as well as histopathological lesions in some cats up to 8 weeks post infection. (Relapsing bacteremia after blood transmission of Bartonella henselae-infected cats, Kordick et al., American Journal of Veterinary Research, 1997, 58: pages 492-497). Other clinical signs in kittens experimentally infected with Bartonella henselae have included lethargy and anorexia. (Clinical disease in kittens inoculated with the pathogenic strain of Bartonella henselae, Mikolajczyk et al., AJVR, volume 61, Number 4, April 2000, page 378). Another interesting observation is that kittens infected with Bartonella henselae have experienced two episodes of clinical signs as opposed to adult cats infected with Bartonella henselae having experienced only one episode of clinical signs. (Id).

There are reports of a broad spectrum of acute and chronic disease syndromes in cats caused by infection with B. henselae, with widespread involvement of many organ systems. There is documented evidence of a case of fatal endocarditis, fatal vegetative endocarditis, and increased frequency of disease in sick seropositive cats leading to stomatitis, kidney disease, urinary tract infections, infection with feline corona virus, and infection with feline spumavirus. Also, B. henselae has been shown to induce reproductive failure, lymphadenopathy, transient central nervous system (CNS) dysfunction, and transient anemia in cats. Histopathology supports the potential role of B. henselae in chronic diseases such as peripheral lymph node and splenic follicular hyperplasia, lymphocytic cholangitis/pericholangitis, lymphocytic hepatitis, lymphocytic plasmacytic myocarditis, and intestinal lymphocytic nephritis. Data also support uveitis (infection of the eyes) in cats following natural exposure or experimental inoculation. Other clinical symptoms reported include fever, CNS involvement, sluggishness, lethargy, anorexia and skin lesions. Therefore, vaccination of cats may serve to prevent infection and potential disease.

It has been well-documented in the literature that there is a strong immune response to infection with Bartonella henselae. (Identification of Bartonella-specific imunodominant antigens recognized by the feline humoral immune system, Freeland et al, Clinical and Diagnostic Immunology, July 1999, pages 558-566). However, the pathogenesis of Bartonella henselae in cats is not clearly understood. A complicating factor in the detection of Bartonella henselae is that cats naturally infected with Bartonella henselae commonly have periods of recurring bacteremia that may last months to years without causing clinical disease during those periods. (Clinical disease in kittens inoculated with the pathogenic strain of Bartonella henselae, Mikolajczyk et al., AJVR, volume 61, Number 4, April 2000, page 375).

In its normal life cycle, B. henselae is probably transmitted from cat fleas (Ctenocephalides felis) to cats. Specifically, viable B. henselae present in the flea feces are probably mechanically transmitted to cats by introduction of infected flea feces either intradermally (self-inoculation by scratching) or orally. Humans are incidental hosts of the bacteria but are susceptible to more serious disease than in the feline natural host. Since the transmission of B. henselae from cats to humans has been demonstrated, an interruption of this process by preventing infection in the feline host can potentially decrease the risk of infection in humans. Therefore, vaccination of felines may have an impact on human public health and will serve to decrease the chance of transmission from infected cats to humans and ultimately serving to increase protection of the human population against infection and disease by B. henselae. Therefore, vaccination of cats for Bartonella is desirable to prevent transmission to humans.

U.S. Pat. No. 5,958,414 (hereinafter referred to as the '414 patent), filed on Sep. 3, 1997, to Regnery et al, discloses and claims a whole cell formalin inactivated Bartonella henselae and a phosphazine polymer adjuvant as a vaccine. The Background Section of the '414 patent discloses that it is untested which adjuvants will work with Bartonella antigens. The '414 patent discloses a phosphazine polymer adjuvant that produces some site reactions and good overall protection. The '414 patent further discloses that the site reactions could be reduced by lowering the overall/amount concentration of the phosphazine adjuvant.

All of the '414 patent vaccine formulations consisted of whole cell Bartonella henselae grown in Vero cells, inactivated with formalin and adjuvanted with the phosphazine polymer. There is no teaching of any other vaccine formulation and no challenge results of any other vaccine formulation.

U.S. Pat. No. 6,774,123 (hereinafter referred to as the '123 patent), filed on Jul. 28, 1999, to Budowsky et al, teaches, in the Background section, that the use of inactivating agents has been known to affect the activity of immunogens. Many inactivating agents modify immunogens nonspecifically; methods using these agents can therefore be difficult to standardize and apply reproducibly.

Surprisingly, it has been found that the teachings of the '414 patent are not applicable to all Bartonella antigen formulations. Further, surprisingly, it has been found that the choice of inactivating agent is a factor in vaccine efficacy. As well, and surprisingly, the method of growth of Bartonella is a factor in vaccine efficacy.

### SUMMARY OF THE INVENTION

Embodiments of the present invention generally relate to vaccines for inhibiting or preventing Bartonella infection and related methods comprising a Bartonella antigen, grown in broth, inactivated by an inactivating agent other than formalin, and an adjuvant. Various embodiments of the vaccines of the present invention comprise inactivated (killed) whole organisms. In preparation of embodiments of the present invention, various carriers, adjuvants, emulsifiers and the like may be used.

Further, various embodiments comprise multivalent vaccines, vaccines with antigens other than Bartonella antigens.

Yet further embodiments comprise vaccination kits and/or diagnostic kits, to both detect and substantially inhibit or prevent infection. Other embodiments comprise methods of vaccination of felines to substantially inhibit or prevent infection with Bartonella, and yet other embodiments comprise methods of substantially inhibiting or preventing humans from acquiring cat scratch disease from felines.

### DETAILED DESCRIPTION OF THE INVENTION

As used herein, the term “vaccine(s)” means and refers to a product, the administration of which is intended to elicit an immune response(s) that can prevent and/or lessen the severity of one or more infectious diseases. A vaccine may be an immunogenic composition comprising a live attenuated bacteria, viruses or parasites, inactivated (killed) whole organisms, living irradiated cells, crude fractions or purified immunogens, including those derived from recombinant DNA in a host cell, conjugates formed by covalent linkage of components, synthetic antigens, polynucleotides (such as plasmid DNA vaccines), living vectored cells expressing specific heterologous immunogens, or cells pulsed with an immunogen. It may also be a combination of vaccines listed above.

As used herein, the term “antigen” means and refers to a virus, a bacteria, parts of a virus or bacteria, or a foreign protein that acts to stimulate the immune system in an animal. The immune system can be stimulated to cause the white blood cells to attack and destroy the antigen or to produce a protein molecule, which attaches to the antigen and either kills the antigen or makes it inactive. As used herein, the term “antibody” means and refers a protein-containing molecule that an animal's immune system makes that reacts with an antigen to make it inactive.

As used herein, the term “vaccine strain” means and refers to a viral or bacterial strain suitable for use in an immunogenic composition or vaccine. A “vaccine strain” can comprise, but is not necessarily limited to, a non-pathogenic strain or relatively non-pathogenic strain, a killed strain, and/or an attenuated strain.

As used herein, the term “lyophilize,” and conjugations thereof, means and refers to, to dry, freeze dry. As used herein, the term “animal origin” means and refers to directly or indirectly originating from animals. Likewise, the term “non-animal origin” means and refers to not originating directly or indirectly from animals.

As used herein, the term “stabilize,” and conjugations thereof, means and refers to make or hold stable, firm, steadfast and to maintain at about a given or substantially unfluctuating level, about a given or substantially unfluctuating quality and about a given or substantially unfluctuating quantity. However, it is understood that some fluctuation in the level, quality, and/or quantity of the stabilized composition may be encountered. Embodiments of the present invention are intended to encompass stabilizers that allow such fluctuations. As well, stabilizers are often referred to or used as a dry stabilizer, a bulk stabilizer, a cryoprotectant, a thermo-stabilizer, an osmoprotectant, a desiccation protectant, and the like. Such terms are specifically meant to be included within the stabilizers of the present invention.

As used herein, the term “protein” means and refers to a molecular chain of amino acids. A protein is not of a specific length and can, if required, be modified in vivo or in vitro, by, e.g. glycosylation, amidation, carboxylation or phosphorylation. Inter alia, peptides, oligopeptides and polypeptides are included within the definition. A protein or peptide can be of biologic and/or synthetic origin.

As used herein, the term “nucleic acid” means and refers to a molecular chain of deoxyribo- or ribonucleic acids. A nucleic acid is not of a specific length, therefore polynucleotides, genes, open reading frames (ORF's), probes, primers, linkers, spacers and adaptors are included within the definition. A nucleic acid can be of biologic and/or synthetic origin. The nucleic acid may be in single-stranded or double stranded form. The single strand may be in sense or anti-sense orientation. Also included within the definition are modified RNAs or DNAs. Modifications in the bases of the nucleic acid may be made, and bases such as Inosine may be incorporated. Other modifications may involve, for example, modifications of the backbone.

As used herein, a pharmaceutically acceptable carrier is understood to be a compound that does not adversely affect the health of the animal or organism to be vaccinated, at least not to the extent that the adverse effect is worse than the effects seen when the animal is not vaccinated. A pharmaceutically acceptable carrier can be e.g. sterile water or a sterile physiological salt solution. In a more complex form the carrier can be, for example, a buffer.

As used herein, the term “carbohydrate” means and refers to the four major groups of saccharides: mono- , di-, oligo-, and poly-saccharides.

As used herein, the term “feline” means and refers to any animal of or pertaining to the genus Felis, or family Felidae, cat family, such as, but not limited to, a cat, a lion, a tiger, a mountain lion, a puma, a bobcat, and the like.

As used herein, the term “vaccine kit” means and refers to a kit for delivering a vaccine to an organism. Parts of a vaccine kit may, but is not required to, comprise needles(s), syringe(s), diluent, antigen, adjuvant, instructions, cloths, and/or the like.

Accordingly, various embodiments of the present invention generally relate to a use of Bartonella antigens for the manufacture of a vaccine suitable for the prevention of Bartonella infection in felines and related methods of use of such vaccines.

In preparing a vaccine antigen of the present invention, cells of Bartonella are introduced into a suitable culture medium, which is incubated at a temperature favoring the growth of the organism. Tryptose phosphate broth (TPB) or Brucella broth may be used for propagation of the organism. However, those skilled in the art would readily be able to determine what other broth media may be used for growth.

The growth of the Bartonella in the broth may be varied. In general, propagation temperatures of 35° C. to 39° C. are favorable. However, the particular temperature will be dependant upon the particular medium chosen and will vary accordingly. Further, the time of incubation may vary. In general, incubation times can vary from a few hours to a few days. Subsequently, the cells can be harvested from the culture medium with or without concentration. Surprisingly, it has been found that growth of the Bartonella in a Brucella broth produces bacteria antigen at high titer sufficient for production of immunogenically effective antigen for various embodiments of the present invention.

In addition to an immunogenically effective amount of Bartonella antigen, the vaccine may contain a pharmaceutically acceptable carrier or diluent. Examples of pharmaceutically acceptable carriers or diluents useful in the present invention include stabilizers such as SPGA (sucrose, phosphate, glutamate, and human albumin as a stabilizer), carbohydrates (e.g., sorbitol, mannitol, starch, sucrose, glucose, and dextran), proteins such as albumin or casein, protein containing agents such as bovine serum or skimmed milk, and buffers (e.g. phosphate buffer). Other stabilizers appropriate for use in various embodiments of the present invention include non-animal origin stabilizers as found in U.S. provisional application 60/537,455, filed on Jan. 15, 2004.

Optionally, one or more compounds having adjuvant activity may be added to the vaccine. Suitable adjuvants are, for example, aluminium hydroxide, phosphate or oxide, oil-emulsions, (such as Paraffin oil, low viscosity oil-emulsion adjuvant or mineral based oil-emulsion adjuvant), saponins or vitamin-E solubilisate. Such adjuvants are especially useful in inactivated and killed embodiments of the vaccine. However, adjuvants may be used in all embodiments.

Various embodiments further comprise one or more emulsifiers. It is most common, that emusifiers are used in an inactivated embodiment of the present invention. However, such emulsifiers are optional and dependent upon the vaccine formulation, not a required component.

The useful dosage to be administered will vary depending on the age, weight and mode of administration. A suitable dosage can be, for example, about 10⁴ cfu eqivalents/animal to about 10¹⁰ (colony forming unit(equivalents)/animal). However, dosage amounts may vary.

For administration, embodiments of the present invention can be given intranasally, intradermally, subcutaneously intramuscularly, or orally.

Bartonella antigens suitable for use in various embodiments of the present invention comprise inactivated whole cells, i.e., bacterins, live-attenuated bacteria, and relevant antigens capable of inducing a protective immune response in inoculated animals. Moreover, all species of Bartonella and related organisms are contemplated within the scope of the present invention. Suitable, non-limiting examples comprise B. henselae, B. elizabethae, B. grahamii, B. vinsonii subsp. arupensis, B. vinsonii subsp. berkhoffi, B. grahamii, B. washoensis, B. koehlerae, B. alsatica, B. doshiae, B. peromysci, B. talpae, B. taylorii, B. tribocorum, B. bovis, B. schoenbuchensis and/or the like.

For the inactivated embodiments, the inactivating agent selected has been shown to be important. A preferred inactivating agent is betapropiolactone (BPL). However, a number of other inactivating agents such as, but not limited to binary ethylenimine and acetyl ethylenimine have been shown to work with varying degrees of efficacy. Suitable concentrations of inactivating agent are about 0.01 to about 0.5%.

Various embodiments may also comprise additional antigens. Suitable examples of additional antigens for use with embodiments of the present invention include, but are not limited to species of Coxiella, Ehrlichia, Brucella Chlamydia, Campylobacter, Helicobacter, Leptospira, Borrellia, and the like. Also, viral antigens including calcivirus, panleukpenia virus, rhinotracheitis virus, rabies, and the like.

In further embodiments, a vaccine kit is disclosed. In varying embodiments, a vaccine kit of the present invention may be sold alone. In other embodiments, a vaccine kit of the present invention is sold with the diagnostic found in U.S. application Ser. No. 10/176,735, filed on Jun. 21, 2002.

The vaccine according to the invention may be administered to the cats by parenteral administration, e.g., intra-muscular or subcutaneous injection, or via intra-nasal, oral, intra-ocular or intra-tracheal administration.

In various embodiments, vaccines of the present invention contain about 10² to about 10¹⁰ CFU equivalents or 1 to 500 μg of subunit antigen per dose. In various other embodiments, vaccines of the present invention contain about 10⁴ to about 10⁸ CFU equivalents or 1 to 500 μg of subunit antigen per dose. However, CFU equivalents per dose may vary.

The appropriate time for vaccination can vary and may be given at any point in the lifespan of the feline. However, as an intent of vaccination is to stimulate a protective humoral and/or cellular immune response in healthy animals to targeted disease organisms, it is recommended that vaccination begin at an early age. A suitable vaccination regime for the vaccine of the present invention comprises a first and second vaccination at 6-9 weeks and 9-17 weeks of age, respectively. Optionally, followed by booster vaccinations as needed and/or desired. Though vaccination usually begins between 6-9 weeks-of-age, the first vaccination can be administered as early as 3-weeks-of-age. However, at younger ages vaccine efficacy is at least partially dependent on the presence and concentration of Bartonella-specific maternal antibodies.

Further embodiments of the present invention generally relate to methods of vaccination of felines to substantially inhibit and/or prevent infection by Bartonella. In an embodiment, the Bartonella is Bartonella henselae. However, other species or strains of Bartonella can also be used, as has been previously specified.

Further embodiments of the present invention are generally related to the vaccination of a feline to prevent transmission of cat scratch disease to humans comprising the step of vaccinating felines with a Bartonella antigen whereby the vaccination substantially inhibits and/or prevents the transmission of cat scratch bacteria from the feline to the human. In an embodiment, the feline is a cat.

While the invention has been described in connection with specific embodiments thereof, it will be understood that it is capable of further modifications and the appended Claims are intended to cover any variations, uses, or adaptations of the invention following, in general, the principles of the invention and including such departures from the present disclosure as come within known or customary practice within the art to which the invention pertains and as may be applied to the essential features hereinbefore set forth whether now existing or after arising. Further, while embodiments of the invention have been described with specific dimensional characteristics and/or measurements, it will be understood that the embodiments are capable of different dimensional characteristics and/or measurements without departing from the principles of the invention and the appended claims are intended to cover such differences. Furthermore, all patents, patent applications, articles, and other publications mentioned herein are herby incorporated by reference.

Examples

Seven studies were conducted with seven different vaccine preparations.

Vaccine Preparation

A study was first performed to determine the proper growth medium and inactivating agent for the vaccine, Study 1a and Study 1b.

Study 1a:

The vaccines of Study 1a, were prepared by culturing the bacteria in a broth medium in shake flasks for 7 days at 37° C. in a 12% CO₂ environment. The antigen dose was harvested from the culture broth and inactivated with BPL. The resulting antigen was washed 2× by centrifugation, resuspended to final antigen dose in Dulbecco's PBS, and frozen at −20° C. until formulation. The final vaccine dose was formulated with an equal part of a non-oil adjuvant.

Study 1b:

The vaccines of Study 1b, were prepared by culturing two (2) lots of the bacteria. One lot was cultured in a broth medium in shake flasks for 7 days at 37° C. in a 12% CO₂ environment. The other lot was cultured in an agar medium for 7 days at 37° C. in a 12% CO₂ environment. The antigen dose was harvested from the culture broth and inactivated with formalin. The resulting antigen was washed 2× by centrifugation, resuspended to final antigen dose in Dulbecco's PBS, and frozen at −20° C. until formulation. The final vaccine dose was formulated with an equal part of a non-oil adjuvant.

The formalin inactivated vaccine preparations were tested in cats. Cats were vaccinated twice, three weeks apart, by the subcutaneous (SC) route. Four weeks after the 2^{nd} vaccination, all cats were challenged by SC inoculation with live B. henselae. In lot 1, 3/6 were protected; and in, lot 2, 2/6 were protected. In light of this data, a decision was made to abandon the formalin inactivated vaccine in favor of the BPL inactivated vaccine.

| Lot # | Vaccine | Protected |
|---|---|---|
| 1 | agar/for malin | 3/6 |
| 2 | broth for malin | 2/6 |

Study 2:

The vaccines of Study 2, were prepared by culturing the bacteria in a broth medium in shake flasks for 78 hours at 37° C. in a 5% CO₂ environment. The antigen dose was harvested from the culture broth and inactivated with BPL. The antigen was then concentrated 10-fold with a hollow cartridge. The resulting antigen was washed 3× by centrifugation, resuspended to final antigen dose in Dulbecco's PBS, and frozen at −20° C. until formulation. The final vaccine dose was formulated with an equal part of a non-oil adjuvant.

Study 3:

The vaccines of Study 3, were prepared by culturing the bacteria in a broth medium in 20-L fermentation vats for 31.5 hours under microaerophilic conditions. The antigen dose was harvested from the culture broth and inactivated with BPL. The antigen was then concentrated 5-fold with a hollow fiber cartridge. The resulting antigen was washed 10× by centrifugation, resuspended to final antigen dose in 50 mM PBS, and frozen at −20° C. until formulation. The final vaccine dose was formulated with an equal part of a non-oil adjuvant.

Study 4:

The vaccines of Study 4 were prepared by culturing the bacteria in a broth medium in 20-L fermentation vats for 31.5 hours under microaerophilic conditions. The antigen dose was harvested from the culture broth and inactivated BPL. The antigen was then concentrated 5-fold with a hollow fiber cartridge. The resulting antigen was washed 10× by centrifugation, resuspended to final antigen dose in 50 mM PBS, and frozen at −20° C. until formulation. The final vaccine dose was formulated with an equal part of a non-oil adjuvant.

Study 5:

The vaccines of Study 5, were prepared by culturing the bacteria in a broth medium. The antigen dose was harvested from the culture broth and inactivated with BPL. The antigen was then concentrated 10-fold with a hollow fiber cartridge. The resulting antigen was washed 3× by centrifugation, resuspended to final antigen dose in Dulbecco's PBS, and frozen at −20° C. until formulation. The final vaccine dose was formulated with an equal part of a non-oil adjuvant.

Study 6:

The vaccines of Study 6, were prepared by culturing the bacteria in a broth medium in 20-L fermentation vats. The antigen dose was harvested from the culture broth and inactivated BPL. The antigen was then concentrated 5-fold with a hollow fiber cartridge. The resulting antigen was washed 10× by centrifugation, resuspended to final antigen dose in 50 mM PBS, and frozen at −20° C. until formulation. The final vaccine dose was formulated with an equal part of a non-oil adjuvant.

Study 7:

The vaccines of Study 7, were prepared in three lots. Lots 1, 2, and 3 were prepared by culturing the bacteria in a broth medium in 20-L fermentation vats for 28.25 to 46.25 hours under microaerophilic conditions. Also for lots 1, 2, and 3, the antigen dose was harvested from the culture broth and inactivated BPL. For lots 1 and 2, the antigen was then concentrated 22.5-fold with a hollow fiber cartridge. The resulting antigen was washed by batch washing and diafiltration, resuspended to final antigen dose in 50 mM PBS, and frozen at −20° C. until formulation. For lot 3, the antigen was then concentrated 5-fold with a hollow fiber cartridge. The resulting antigen was washed 10× by centrifugation, resuspended to final antigen dose in 50 mM PBS, and frozen at −20° C. until formulation. The final vaccine dose for lot 1 was formulated with a colorant, preservative agents, and then with an equal part of a non-oil adjuvant. The final vaccine dose for lot 2 was formulated with a colorant, a preservative agent, and then with an equal part of a non-oil adjuvant. The final vaccine dose for lot 3 was formulated with an equal part of non-oil adjuvant.

Challenge Study

A challenge study was then performed the results of which can be found in Table 1, also FIG. 1.

Study 1 and 2:

In Study 1a and 2, groups of six cats were vaccinated. Cats were vaccinated twice, three weeks apart, by the subcutaneous (SC) route. Four weeks after the 2nd vaccination, all cats were challenged by SC inoculation with live B. henselae. In both Study #1 and #2, 100% of the vaccinated cats (6/6) were protected against infection after challenge. In Study 1, 100% of the challenge control cats (6/6) became infected. In Study 2, 83% of the challenge control cats (5/6) became infected.

Study 3 and 4:

In Study 3 and 4, groups of six cats were vaccinated. Cats were vaccinated twice, three weeks apart, by SC injection of vaccine. In study 3, cats were challenged five weeks after the 2nd vaccination, while in study 4 cats were challenged three months after the 2nd vaccination. Studies 3 and 4 shared the challenge control group in order to minimize use of animals. In both studies 3 and 4, 67% of the vaccinated cats (4/6) were protected against infection after challenge. Also, 100% of the challenge control cats (6/6) shared by these two studies became infected.

Study 5:

In Study 5, six cats were vaccinated. Cats were vaccinated twice, three weeks apart, by the SC route. Cats were challenged four weeks after the 2nd vaccination. In this study, 83% of the vaccinated cats (5/6) were protected against infection after challenge, while 100% of the challenge control cats (6/6) became infected.

Study 6:

In Study 6, six cats were vaccinated. Cats were vaccinated twice, three weeks apart, by SC injection of vaccine. Cats were challenged four weeks after the 2nd vaccination. In this study, 67% of the vaccinated cats (4/6) were protected against infection after challenge, while 100% of the challenge control cats (6/6) became infected.

Study 7:

In Study 7, 18 cats were vaccinated, six with each of lot 1, 2, and 3. Cats were vaccinated twice, three weeks apart, by the SC route. Cats were challenged four weeks after the 2nd vaccination. In this study, 94% of the vaccinated cats (17/18) were protected against infection after challenge, while 100% of the challenge control cats (6/6) became infected.

TABLE 1
Summary of seven vaccine evaluation studies.

| | | | No. cats | No. of cats |
|---|---|---|---|---|
| | | | bacteremic | w/vaccine |
| | No. of | | after | site |
| Study | cats | Vaccine | challenge | reactions |
| 1 | 6 | Adjuvanted inactivated | 0/6 (0%) | 3/6 |
| | | whole-cell B. henselae | | |
| | | vaccine | | |
| 1 | 6 | None (challenge control | 6/6 (100%) | N/A |
| | | group) | | |
| 2 | 6 | Adjuvanted inactivated | 0/6 (0%) | 1/6 |
| | | whole-cell B. henselae | | |
| | | vaccine | | |
| 2 | 6 | None (challenge control | 5/6 (83%) | N/A |
| | | group) | | |
| 3 | 6 | Adjuvanted inactivated | 2/6 (33%) | 3/6 |
| | | whole-cell B. henselae | | |
| | | vaccine | | |
| 4 | 6 | Adjuvanted inactivated | 2/6 (33%) | 1/6 |
| | | whole-cell B. henselae | | |
| | | vaccine | | |
| 3, 4 | 6 | None (challenge control | 6/6 (83%) | N/A |
| | | group-shared by studies | | |
| | | #3 and #4) | | |
| 5 | 6 | Adjuvanted inactivated | 1/6 (17%) | 6/6 |
| | | whole-cell B. henselae | | |
| | | vaccine | | |
| 5 | 6 | None (challenge control | 6/6 (100%) | N/A |
| | | group) | | |
| 6 | 6 | Adjuvanted inactivated | 2/6 (33%) | 0/6 |
| | | whole-cell B. henselae | | |
| | | vaccine | | |
| 6 | 6 | None (challenge control | 6/6 (100%) | N/A |
| | | group) | | |
| 7 | 6 | Adjuvanted inactivated | 0/6 (0%) | 4/6 |
| | | whole-cell B. henselae | | |
| | | vaccine | | |
| 7 | 12 | Adjuvanted inactivated | 1/12 (8%) | 3/12 |
| | | whole-cell B. henselae | | |
| | | vaccine | | |
| 7 | 6 | None (challenge control | 6/6 (100%) | N/A |
| | | group) | | |

1. A vaccine composition for protecting a mammal from infection with Bartonella-comprising:
an immunogenically effective amount of a whole cell inactivated Bartonella antigen, wherein the Bartonella has not been inactivated with formalin and a pharmaceutically acceptable carrier or diluent.

2. The vaccine composition of claim 1, further comprising an adjuvant.

3. The vaccine composition of claim 1, further comprising a stabilizer.

4. The vaccine composition of claim 3, wherein the composition is lyophilized.

5. The vaccine composition of claim 1, wherein the antigen is grown in a broth.

6. A method for protecting a mammal from infection with Bartonella, comprising the steps of administering the composition of claim 1 to the feline.
